(19) United States
(12) Patent Application Publication
(10) Pub. No.: US 2004/0248846 A1
(43) Pub. Date: Dec. 9, 2004

(54) Intranasal administration of triptans

(75) Inventors: Steven C. Quay, Edmonds, WA (US); Zenaida O. Go, Clifton, NJ (US)

Correspondence Address: PAUL G. LUNN, ESQ. NASTECH PHARMACEUTICAL COMPANY, INC., 3450 MONTE VILLA PARKWAY, BOTHELL, WA, 98021-8906, US

(73) Assignee: Nastech Pharmaceutical Company Inc.

(21) Appl. No.: 10/824,452

(22) Filed: Apr. 14, 2004

Related U.S. Application Data
(60) Provisional application No. 60/464,671, filed on Apr. 22, 2003.

(51) Int. Cl. A61K 31/724; A61K 31/405

(52) U.S. Cl. 514/58; 514/419

(57) ABSTRACT

Methods for treating migraine and cluster headaches by improving the transmucosal Tₘₐₓ and Cₘₐₓ of triptans are disclosed. The intranasal compositions are comprised of water, a triptan and an absorption enhancer, for example, α-cyclodextrin.

This claims priority under 35 U.S.C. §119(e) of United States Provisional Application No. 60/464,671 filed Apr. 22, 2003, the entire contents of which are incorporated herein by reference.

### BACKGROUND

The teachings of all of the references cited herein are incorporated herein by reference.

The current therapy to treat migraine headaches include the administration of serotonin 5-HT 1B/1D receptor agonists collectively called triptans. Sumatriptan was the first of these compounds to be developed, and offered improved efficacy and tolerability over ergot-derived compounds. The development of sumatriptan was quickly followed by a number of ‘second generation’ triptans including zolmitriptan, naratriptan, and rizatriptan. Intuitively, the expectation was that the nasal spray would prove more rapid and efficacious than oral or subcutaneous routes of administration. However, recent polls of migraineurs (people who suffer from migraines more than twice a month) suggest that more lipophilic formulations of other triptans, given orally, are still preferred over the sumatriptan nasal spray (IMITREX®). While these alternative triptans have slightly different binding affinities for the various 5HT receptors, the single guiding factor in success with these formulations seems to be the speed of action. A solution to the problem requires onset of action of the drug in less than 30 minutes, more preferentially within 10 minutes, of application.

Thus, there is a need for an intranasal triptan formulation that has an increased speed of action over those present in the prior art.

### BRIEF DESCRIPTION OF THE DRAWINGS

FIG. 1 shows the Mean tₘₐₓ for sumatriptan nasal spray of the present invention as shown in Example 3, hereinafter referred to as (Nastech Nasal’) Nasal IMITREX®, and Oral IMITREX® The mean Cₘₐₓ (total area) for Nastech's nasal formulation at 5 mg was 2.65 ng/ml and the mean Cₘₐₓ (total area) for the marketed formulation of IMITREX® at 5 mg was 3.28 ng/ml. The mean Cₘₐₓ (total area) values for the marketed 20 mg nasal IMITREX® and the mean Cₘₐₓ (total area) for the marketed 25 mg oral tablet were 10.07 and 14.39 ng/ml, respectively

FIG. 2 shows the mean concentration of sumatriptan for 5 mg of Nastech Nasal and 5 mg of intranasal IMITREX® after 20 minutes

FIG. 3 shows the mean absorption rate in minutes for the sumatriptan formulation of Nastech Nasal and IMITREX® products.

FIG. 4 shows the mean Cmax (ng/mL) of Nastech Nasal and IMITREX® sumatriptan nasal spray (20 minutes pK).

FIG. 5 shows the mean tmax (min) for 5 mg Nastech Nasal and 5 mg IMITREX® nasal spray.

FIG. 6 shows the tmax (min) 5 mg Nastech Nasal and 5 mg IMITREX® nasal spray (60 minute pK).

### DESCRIPTION OF THE INVENTION

The present invention fills this need by providing for an aqueous triptan formulation suitable for intranasal administration of a triptan comprised of a triptan, water, and an absorption enhancer wherein the triptan formulation has a tmax in serum of less than 15 minutes after intranasal administration of a sufficient amount of the formulation to deliver 5 mg of the triptan to the nose.

In another embodiment of the present invention an aqueous triptan formulation is provided suitable for intranasal administration of a triptan comprised of a triptan, water, and an absorption enhancer wherein the triptan reaches a mean plasma concentration of at least 1.5 ng-1.8 ng-2.0 ng of triptan per milliliter of plasma within 20 minutes after intranasal administration of a sufficient amount of the formulation to deliver 5 mg of the triptan to the nose.

The present invention still further provides for an aqueous triptan formulation suitable for intranasal administration of a triptan comprised of a triptan, water, and an absorption enhancer wherein the triptan formulation has a mean partial areas under the curve of triptan for the first 20 minutes after intranasal administration of a sufficient amount of the formulation to deliver 5 mg of the triptan to the nose of at least 25-30 ng per minute per mL of serum.

In another embodiment of the present invention an aqueous triptan formulation is provided suitable for intranasal administration of a triptan comprised of a triptan, water, and an absorption enhancer wherein the triptan formulation has a mean absorption rate of less than 20 minutes, more preferably less than 15 minutes after intranasal administration of a sufficient amount of the formulation to deliver 5 mg of the triptan to the nose.

In yet another embodiment, the present invention provides for an aqueous triptan formulation suitable for intranasal administration of a triptan comprised of a triptan, water, and an absorption enhancer wherein the triptan formulation has a mean Cₘₐₓ of at least 1.5 ng of triptan per mL of serum 20 minutes after intranasal administration of a sufficient amount of the formulation to deliver 5 mg of the triptan to the nose.

The formulations can contain any triptan including naratriptan, almotriptan, frovatriptan, rizatriptan, zolmitriptan and sumatriptan. A preferred triptan is sumatriptan.

It has been surprisingly discovered that the addition of an absorption enhancer, in particular a chitosan or a cyclodextrin, in particular alpha-cyclodextrin greatly improves the speed that a triptan enters into the blood serum upon intranasal administration of an aqueous triptan formulation containing a cyclodextrin. In a preferred formulation the cyclodextrin is present at a concentration of about 5% wt/wt, and the cyclodextrin is alpha-cyclodextrin. Another cylodextrin, which can be used is methyl-β-cyclodextrin.

In alternative formulations chitosan or a chelating agent may also be added. If chitosan is added a preferred concentration of chitosan is 0.4% wt/wt. In another formulation, the aqueous formulation is comprised of water, one or more triptans, a cyclodextrin and a chelating agent. Preferably the chelating agent is ethylenediaminetetraacetic acid (EDTA) at a concentration of about 0.1% wt/wt and the cyclodextrin is alpha-cyclodextrin at a concentration of about 5% wt/wt.

In another embodiment, we supply mucolytic agents to further decrease the viscosity of the nasal mucosa. These include n-acetyl-cysteine, propyl gallate and cysteine methionine dimers that compete by mass action for sulfhydryls and disulfide bonds in the mucous polymer. As disulfide bonds are broken, the overall viscosity of the mucous is reduced significantly. Other forms of secondary structure in the mucosa are cooperative hydrogen bonded structures involving polysaccharide and aminosaccharide chains. This cooperative hydrogen bonding is reduced with the chaotropes, and it is actually the free energy of the increased entropy from the hydrogen bonded ordered structures as they become randomized that is contributed as a pleasant sensation of warmth in the nostril during application of the formulations of this invention.

Extracellular diffusive entry in the nose follows rules related to molecular size, ionicity and lipophilicity. Permeation is inversely proportionate with molecular weight, but may be detected at up to 20 kDa. Preferential permeation occurs for molecules below 1 KDa. In a range of partition coefficients (-Log P) from 0 to 1.0, the perfusion of drug into the CSF increased by almost a factor of 10 as the drug became more lipophilic. Permeation relates best to unionized fraction of permeant, in concert with the pH Partition Theory.

In another aspect, various permeation enhancing agents may be used to improve intranasal uptake. Certain penetration enhancing substances, “enhancers,” that facilitate the transport of solutes across biological membranes are being developed for facilitating mucosal drug delivery. Mucosal penetration enhancers include (a) chelators (e.g., citric acid, salicylates), (b) surfactants (e.g., Tween 80 or Poloxamer 188, (c) chaotropes and solvents (e.g., unsaturated cyclic ureas and Transcutol), (d) bile salts (e.g., sodium deoxycholate, sodium taurocholate), and (e) fatty acids (e.g., oleic acid, short chain mono- and diglycerides). Various mechanisms have been proposed for enhancing mucosal penetration of drugs. These include, for example, reducing the viscosity and/or thickness of the mucin layers that cover mucosal surfaces; facilitating transmembrane transport by increasing the fluidity of the lipid bilayer of membranes; using prodrugs to alter the physicochemical properties (e.g., lipophilicity, stability) of the active substance; facilitating paracellular transport by altering the permeability of the tight junctions in the epithelial cell layer; further overcoming enzymatic barriers; and methods for increasing the thermodynamic activity of candidate drugs.

Therefore, in all its manifestations, the invention is recognizable when embodied in the form of nutriceuticals, pharmaceuticals, and as kits and devices for administration of the inventive compositions, or for use in the inventive methods.

Rizatriptan, zolmitriptan, sumatriptan, naratriptan, almotriptan, frovatriptan, donitriptan, and eletriptan are sold or under study for arrest of migraine pain after its onset. A critical feature of the success of this therapy is the speed and consistency with which relief is obtained.

These drugs are of use in the treatment of chronic paroxysmal headache, cluster headache, migraine headache, basilar migraine, familiar hemiplegic migraine, migraine with and without aura, and of atypical headaches accompanied by autonomic symptoms.

The intranasal formulations of the present invention can be administered using any spray bottle or syringe. An example of a nasal spray bottle is the, “Nasal Spray Pump w/Safety Clip, Pfeiffer SAP # 60548, which delivers a dose of 0.1 mL per squirt and has a diptube length of 36.05 mm. It can be purchased from Pfeiffer of America of Princeton, N.J.

The intranasal triptan formulations of the present invention can be administered for the acute treatment of migraine attacks with or without auras. Single doses of 5, 10, or 20 mg of the triptan nasal sprays of the present invention can be administered in a nostril for the acute treatment of migraines. In the formulation of Example 3, hereinafter referred to as the ‘Nastech Nasal’ formulation), a 10-mg dose may be achieved by the administration of a single 5-mg dose in each nostril.

An unexpected superior property of the Nastech sumatriptan formulation of the present invention containing α-cyclodextrin is the fact that it is tasteless as opposed to the bitter, metallic taste of intranasal IMITREX® formulation.

The following definitions are supplied here in order to provide a clearer explanation of the chemistry and pharmacology described herein.

The term “triptan” as used herein includes compounds designed around an indole ring, with neurotropic activity in suppression of migraine pain. These include the free base form of this compound and its salts of this compound as well as all pharmacologically acceptable analogs, derivatives, and chemically modified forms, including acid addition salts, thereof. In addition to chloride, other acceptable salts are the bromide, the iodide, the sulfuric, the phosphate, the acid phosphate, the lactate, the citrate, the tartarate, the salicylate, the succinate, the maleate, the gluconate, mesylate, and the like. Also included are fatty acid salts of the form “lipophilic ion pairs”, such as the laurate, dodecylate, myristate, palmitate, stearate, coconoate, behinate, oleate, linoleate, linolenate, eicosapentaenoate, eicosahexaenoate, docosapentaenoate, docosahexaenoate, and eicosanoids in general.

“5HT receptor agonists”: In the central nervous system, 5-hydroxytryptamine (5-HT) neurotransmission is mediated through receptors belonging to the 5HT receptor family, which includes Types 1 through 7, and subtypes of those. The 5HT 1D receptor is most closely associated with migraine pain, but the 1A,B and F receptors may also have a role. These agonists are associated with cerebral vasoconstriction. Those trigeminal 5HT receptors located in the “dorsal horn” of the central trigeminal nerve are thought to be key to migraine pain.

“Nasal mucosa” is an epithelium that extends from the nasal atrium to the back of the nasopharyx (pendant the ear on a sagittal view), where the nasal cavity transitions to the oropharynx which leads to the esophagus and trachea. The nasal cavity has been described as a trapezoid about 5 cm in height and 10 cm in depth, with an approximate epithelial area of 150 cm².

Adverse effect”, also termed “side effect” refers to any reaction following administration of a therapeutic that is not a therapeutic effect, except for those symptoms that are associated with the underlying disease.

As used herein “peak concentration (Cₘₐₓ)”, “area under concentration vs. time curve (AUC)”, “time to maximal plasma concentration (tₘₐₓ)” of triptans in blood (or CSF) are pharmacokinetic parameters known to those skilled in the art. The “concentration vs. time curve”, also termed the pK curve, measures the concentration of a drug in a blood serum of a subject over time after administration of a dosage of vitamin to the subject. “Cₘₐₓ” is the mean maximum concentration of therapeutic achieved, usually in blood, following a single dosage of a drug to an experimental population of mammals or test subjects. “tmax” is the mean time to reach maximum concentration of a drug, usually for an experimental population of mammals or test subjects, following administration of a single dosage of drug to each subject.

As used herein, “area under concentration vs. time curve (AUC) of a drug in a blood plasma” is calculated according to the linear trapezoidal rule and with addition of the residual areas. A decrease of 23% or an increase of 30% between two dosages would be detected with a probability of 90% (type II error β=10%). The “delivery rate” or “rate of absorption” is estimated by comparison of the time (tₘₐₓ) to reach the maximum concentration (Cₘₐₓ). Both Cₘₐₓ and tₘₐₓ are analyzed using non-parametric methods. Comparisons of the pharmacokinetics of are performed by analysis of variance (ANOVA). For pairwise comparisons a Bonferroni-Holmes sequential procedure is used to evaluate significance. The dose-response relationship between escalating doses is estimated by regression analysis. For any statistic, P<0.05 is considered significant. Results are given as mean values±SEM.

For intranasal delivery, the triptan intranasal formulations of the present invention combined or coordinately administered with a suitable carrier or vehicle. As used herein, the term “carrier” means a pharmaceutically acceptable liquid. A water-containing liquid carrier can contain pharmaceutically acceptable additives such as acidifying agents, alkalizing agents, buffering agents, antimicrobial preservatives, antioxidants, chelating agents, complexing agents, solubilizing agents, humectants, anti-irritants, solvents, suspending and/or viscosity-increasing agents, tonicity agents, wetting agents or other biocompatible materials. A tabulation of some ingredients listed by the above categories, can be found in the U.S. Pharmacopeia National Formulary, pp. 1857-1859, (1990.). Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other non toxic compatible substances used in pharmaceutical formulations. Wetting agents, emulsifiers and lubricants such as sodium lauryl sulfate and magnesium stearate, TPGS, short chain mono- and diglycerides, as well as coloring agents, release agents, coating agents, sweetening, flavoring and perfuming agents, preservatives and antioxidants can also be present in the compositions, according to the desires of the formulator. Examples of pharmaceutically acceptable anti-oxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like. Examples of pharmaceutically acceptable preservatives include cetrimonium chloride, benzalkonium chloride, disodium EDTA, chlorobutanol, sorbate, sodium borate, sodium perborate, methylpara-hydroxybenzoate, stabilized oxychloro complex (SOC), polyquartemium-1 (Polyquad), or benzododecinium bromide. The amount of active ingredient that can be combined with the carrier materials to produce a single dosage form will vary depending upon the particular mode of administration.

Within the mucosal delivery compositions and methods of the invention, various permeation-enhancement agents are employed which enhance delivery of receptor agonist into or across a mucosal surface. In this regard, delivery of a receptor agonist (or receptor agonists) across the mucosal epithelium can occur by several possible pathways as described earlier.

As used herein, “absorption enhancer” include agents which enhance the release or solubility (e.g., from a formulation delivery vehicle), diffusion rate, penetration capacity and timing, uptake, residence time, stability, effective half-life, peak or sustained concentration levels, clearance and other desired mucosal delivery characteristics (e.g., as measured at the site of delivery, or at a selected target site of activity such as the bloodstream or central nervous system) of receptor agonist or other biologically active compound(s). Enhancement of mucosal delivery can thus occur by any of a variety of mechanisms, for example by increasing the diffusion, transport, persistence or stability of receptor agonist, increasing membrane fluidity, modulating the availability or action of calcium and other ions that regulate intracellular or paracellular permeation, solubilizing mucosal membrane components (e.g., lipids), changing non-protein and protein sulffhydryl levels in mucosal tissues, increasing water flux across the mucosal surface, modulating epithelial junctional physiology, reducing the viscosity of mucus overlying the mucosal epithelium, reducing mucociliary clearance rates, and other mechanisms.

The nasal mucosa contains hydrolytic enzymes, such as lipases and proteases, which must be overcome. This enzymatic “barrier” can be dampened by administering enzyme inhibitors that prevent or at least lessen the extent of degradation.

The level of permeability enhancers used in a formulation must be carefully controlled. Effective permeabilizer concentrations within the epithelium are not easily maintained in vivo. Too little carrier, or carrier concentrations transient, can be ineffective. Too much carrier, or carrier concentrations persistent, may result in irritability.

While the mechanism of absorption promotion may vary with different intranasal absorption agents of the invention, useful reagents in this context will not substantially adversely affect the mucosal tissue and will be selected according to the physicochemical characteristics of the particular receptor agonist or other active or delivery-enhancing agent. In this context, delivery-enhancing agents that increase penetration or permeability of mucosal tissues will often result in some alteration of the protective permeability barrier of the mucosa. For such permeation-enhancement agents to be of value within the invention, it is generally desired that any significant changes in permeability of the mucosa be reversible within a time frame appropriate to the desired duration of drug delivery. Furthermore, there should be no substantial, cumulative toxicity, nor any permanent deleterious changes induced in the barrier properties of the mucosa with long-term use.

Within certain aspects of the invention, absorption enhancers for coordinate administration or combinatorial formulation with receptor agonist of the invention are selected from small hydrophilic molecules, including but not limited to, dimethyl sulfoxide (DMSO), dimethylformamide, ethanol, propylene glycol, and the 2-pyrrolidones. Alternatively, long-chain amphipathic molecules, for example, deacylmethyl sulfoxide, azone, sodium laurylsulfate, oleic acid, and the bile salts, may be employed to enhance mucosal penetration of the receptor agonist. In additional aspects, surfactants (e.g., polysorbates) are employed as adjunct compounds, processing agents, or formulation additives to enhance intranasal delivery of the receptor agonist. These penetration-enhancing agents typically interact at either the polar head groups or the hydrophilic tail regions of molecules, which comprise the lipid bilayer of epithelial cells lining the nasal mucosa. Interaction at these sites may have the effect of disrupting the packing of the lipid molecules, increasing the fluidity of the bilayer, and facilitating transport of the receptor agonist across the mucosal barrier. Interaction of these penetration enhancers with the polar head groups may also cause or permit the hydrophilic regions of adjacent bilayers to take up more water and move apart, thus opening the paracellular pathway to transport of the receptor agonist. In addition to these effects, certain enhancers may have direct effects on the bulk properties of the aqueous regions of the nasal mucosa. Agents such as DMSO, polyethylene glycol, and ethanol can, if present in sufficiently high concentrations in delivery environment (e.g., by pre-administration or incorporation in a therapeutic formulation), enter the aqueous phase of the mucosa and alter its solubilizing properties, thereby enhancing the partitioning of the receptor agonist from the vehicle into the mucosa.

Additional mucosal absorption enhancers that are useful within the coordinate administration and processing methods and combinatorial formulations of the invention include, but are not limited to, mixed micelles; enamines; nitric oxide donors (e.g., S-nitroso-N-acetyl-DL-penicillamine, NOR1, NOR4—which are preferably co-administered with an NO scavenger such as carboxy-PITO or doclofenac sodium); sodium salicylate; glycerol esters of acetoacetic acid (e.g., glyceryl-1,3-diacetoacetate or 1,2-isopropylideneglycerine-3-acetoacetate); and other release-diffusion or intra- or trans-epithelial penetration-promoting agents that are physiologically compatible for mucosal delivery. Other absorption-promoting agents are selected from a variety of carriers, bases and excipients that enhance mucosal delivery, stability, activity or trans-epithelial penetration of the receptor agonist. These include, inter alia, cyclodextrins and α- or β-cyclodextrin derivatives. These compounds, optionally conjugated with one or more of the active ingredients and further optionally formulated in an oleaginous base, enhance bioavailability in the mucosal formulations of the invention. Yet additional absorption-enhancing agents adapted for mucosal delivery include medium-chain fatty acids, including mono- and diglycerides (e.g., sodium caprate—extracts of coconut oil, Capmul), and triglycerides (e.g., amylodextrin, Estaram 299, Miglyol 810).

The mucosal therapeutic and prophylactic compositions of the present invention may be supplemented with any suitable absorption enhancer that facilitates absorption, diffusion, or penetration of receptor agonist across mucosal barriers. The penetration promoter may be any promoter that is pharmaceutically acceptable. Thus, in more detailed aspects of the invention compositions are provided that incorporate one or more penetration-promoting agents selected from sodium salicylate and salicylic acid derivatives (acetyl salicylate, choline salicylate, salicylamide, etc.); amino acids and salts thereof (e.g. monoaminocarboxlic acids such as glycine, alanine, phenylalanine, proline, hydroxyproline, etc.; hydroxyamino acids such as serine; acidic amino acids such as aspartic acid, glutamic acid, etc; and basic amino acids such as lysine etc—inclusive of their alkali metal or alkaline earth metal salts); and N-acetylamino acids (N-acetylalanine, N-acetylphenylalanine, N-acetylserine, N-acetylglycine, N-acetyllysine, N-acetylglutamic acid, N-acetylproline, N-acetylhydroxyproline, etc.) and their salts (alkali metal salts and alkaline earth metal salts).

Also provided as absorption enhancers within the methods and compositions of the invention are substances which are generally used as emulsifiers (e.g. sodium oleyl phosphate, sodium lauryl phosphate, sodium lauryl sulfate, sodium myristyl sulfate, polyoxyethylene alkyl ethers, polyoxyethylene alkyl esters, etc.), caproic acid, lactic acid, malic acid and citric acid and alkali metal salts thereof, pyrrolidonecarboxylic acids, alkylpyrrolidonecarboxylic acid esters, N-alkylpyrrolidones, proline acyl esters, and the like.

Because of the complexity of surfactants usefil in these arts, we offer a more comprehensive list herein.

Suitable surfactants can be ionic or non-ionic surfactants. The surfactant can be any surfactant suitable for use in pharmaceutical compositions. Suitable hydrophilic surfactants can be anionic, cationic, zwitterionic or non-ionic. It should be emphasized that the invention is not limited to the surfactants disclosed here, and that commercially supplied surfactants are commonly impure, or contain ranges of side chains and polar groups. HLB values are given for reference.

A variety of PEG-fatty acid esters have useful surfactant properties. Examples of polyethoxylated fatty acid monoester surfactants include, but are not limited to, PEG 4-100 monolaurate, Crodet L series (Croda), HLB>9; PEG 4-100 monooleate, Crodet O series (Croda), HLB>8; PEG 4-100 monostearate, Crodet S series (Croda), Myrj Series, (Atlas/ICI), HLB>6; PEG 400 distearate, Cithrol 4DS series (Croda), HLB>10; PEG 100, 200, 300 monolaurate, Cithrol ML series (Croda), HLB>10; PEG 100, 200, 300 monooleate, Cithrol MO series (Croda), HLB>10; PEG 400 dioleate, Cithrol 4DO series (Croda), HLB>10; PEG 400-1000 monostearate, Cithrol MS series (Croda), HLB>10; PEG-1 stearate, Nikkol MYS-1EX (Nikko), Coster K1 (Condea), HLB 2; PEG-2 stearate, Nikkol MYS-2 (Nikko), HLB 4; PEG-2 oleate, Nikkol MYO-2 (Nikko), HLB 4.5; PBG-4 laurate, Mapeg® 200 ML (PPG), Kessco® PEG 200 ML (Stepan), LIPOPEG 2L (LIPO Chem.), HLB 9.3; PEG-4 oleate, Mapeg® 200 MO (PPG), Kessco® PEG 200 MO (Stepan), HLB 8.3; PEG-4 stearate, Kessco® PEG 200 MS (Stepan), Hodag 20 S (Calgenc), Nikkol MYS-4 (Nikko), HLB 6.5; PEG-5 stearate, Nikkol TMGS-5 (Nikko), HLB 9.5; PEG-5 oleate, Nikkol TMGO-5 (Nikko), HLB 9.5; PEG-6 oleate, Algon OL 60 (Auschem SpA), Kessco® PEG 300 MO (Stepan), Nikkol MYO-6 (Nikko), Emulgante A6 (Condea), HLB 8.5; PEG-7 oleate, Aigon OL 70 (Auschem SpA), HLB 10.4; PEG-6 laurate, Kessco® PEG 300 ML (Stepan), HLB 11.4; PEG-7 laurate, Lauridac 7 (Condea, HLB 13; PEG-6 stearate, Kessco® PEG 300 MS (Stepan), HLB 9.7; PEG-8 laurate, Mapeg® 400 ML (PPG), LIPOPEG 4DL (Lipo Chem.), HLB 13; PEG-8 oleate, Mapeg® 400 MO (PPG), Emulgante A8 (Condea), HLB 12; PEG-8 stearate, Mapeg® 400 MS (PPG), Myd 45, HLB 12; PEG-9 oleate, Emulgante A9 (Condea), HLB>10; PEG-9 stearate, Cremophor S9 (BASF), HLB>10; PEG-10 laurate, Nikkol MYL-10 (Nikko), Lauridac 10 (Croda), HLB 13; PEG-10 oleate, Nikkol MYO-10 (Nikko), HLB 11; PEG-10 stearate, Nikkol MYS-10 (Nikko), Coster K100 (Condea), HLB 11; PEG-12 laurate, Kessco® PEG 600 ML (Stepan), HLB 15; PEG-12 oleate, Kessco® PEG 600 ML (Stepan), HLB 14; PEG_{—}12 ricinoleate, (CAS # 9004-97-1), HLB>10; PEG-12 stearate, Mapeg® 600 MS (PPG), Kessco® PEG 600 MS (Stepan), HLB 14; PEG-15 stearate, Nikkol TMGS-15 (Nikko), Koster K5 (Condea), HLB 14; PEG-15 oleate, Nikkol TMGO-15 (Nikko), HLB 15; PEG-20 laurate, Kessco® PEG 1000 ML (Stepan), HLB 17; PEG-20 oleate, Kessco® PEG 1000 MO (Stepan), HLB 15; PEG-20 stearate, Mapeg® 1000 MS (PPG), Kessco® PEG 1000 MS (Stepan), Myrj 49, HLB 16; PEG-25 stearate, Nikkol MYS-25 (Nikko) HLB 15; PEG-32 laurate, Kessco® PEG 1540 ML (Stepan), HLB 16; PEG-32 oleate, Kessco® PEG 1540 MO (Stepan), HLB 17; PEG-32 stearate, Kessco(g PEG 1540 MS (Stepan), HLB 17; PEG-30 stearate, Myrj 51, HLB>10; PEG-40 laurate, Crodet L40 (Croda), HLB 17.9; PEG-40 oleate, Crodet 040 (Croda), HLB 17.4; PEG-40 stearate, Myrj 52, Emerest® 2715 (Henkel), Nikkol MYS-40 (Nikko), HLB >10; PEG-45 stearate, Nikkol MYS-45 (Nikko), HLB 18; PEG-50 stearate, Myrj 53, HLB>10; PEG-55 stearate, Nikkol MYS-55 (Nikko), HLB 18; PEG-100 oleate, Crodet 0-100 (Croda), HLB 18.8; PEG-100 stearate, Myrj 59, Arlacel 165 (ICI), HLB 19; PEG-200 oleate, Albunol 200 MO (Taiwan Surf.) HLB>10; PEG-400 oleate, LACTOMUL (Henkel), Albunol 400 MO (Taiwan Surf.), HLB>10, PEG-600 oleate, Albunol 600 MO (Taiwan Surf.), HLB>10.

Polyethylene glycol fatty acid diesters are also surfactants. PEG-fatty acid diesters include, but are not limited to, PEG-4 dilaurate, Mapeg® 200 DL (PPG), Kessco® PEG 200 DL (Stepan), LIPOPEG 2-DL (Lipo Chem.), HLB 7; PEG-4 dioleate, Mapeg® 200 DO (PPG), HLB 6; PEG-4 distearate, Kessco® 200 DS (Stepan), HLB 5; PEG-6 dilaurate, Kessco® PEG 300 DL (Stepan), HLB 9.8; PEG-6 dioleate, Kessco® PEG 300 DO (Stepan), HLB 7.2; PEG-6 distearate, Kessco® PEG 300 DS (Stepan), HLB 6.5; PEG-8 dilaurate, Mapeg® 400 DL (PPG), Kessco® PEG 400 DL (Stepan), LIPOPEG 4 DL (Lipo Chem.), HLB 11; PEG-8 dioleate, Mapeg® 400 DO (PPG), Kessco® PEG 400 DO (Stepan), LIPOPEG 4 DO (Lipo Chem.), HLB 8.8; PEG-8 distearate, Mapeg® 400 DS (PPG), CDS 400 (Nikkol), HLB 11; PEG-10 dipalmitate, Polyaldo 2PKFG, HLB >10; PEG-12 dilaurate, Kessco® PEG 600 DL (Stepan), HLB 11.7; PEG-12 distearate, Kessco® PEG 600 DS (Stepan), HLB 10.7; PEG-12 dioleate, Mapeg(® 600 DO (PPG), Kessco® 600 DO (Stepan), HLB 10; PEG-20 dilaurate, Kessco(® PEG 1000 DL (Stepan), HLB 15; PEG-20 dioleate, Kessco® PEG 1000 DO (Stepan), HLB 13; PEG-20 distearate, Kessco® PEG 1000 DS (Stepan), HLB 12; PEG-32 dilaurate, Kessco® PEG 1540 DL (Stepan), HLB 16; PEG-32 dioleate, Kessco® PEG 1540 DO (Stepan), HLB 15; PEG-32 distearate, Kessco® PEG 1540 DS (Stepan), HLB 15; PEG-400 dioleate, Cithrol 4DO series (Croda), HLB>10; PEG-400 distearate, Cithrol 4DS series (Croda), HLB>10.

Polyethylene glycol fatty acid mono and di-ester mixtures include, but are not limited to, PEG 4-150 mono, dilaurate, Kessco® PEG 200-6000 mono, dilaurate (Stepan); PEG 4-150 mono, dioleate, Kessco® PEG 200-6000 mono, dioleate (Stepan); PEG 4-150 mono, distearate, Kessco® 200-6000 mono, distearate (Stepan).

Polyethylene glycol glycerol fatty acid esters include, but are not limited to, PEG-20 glyceryl laurate, Tagat® L (Goldschmidt), HLB 16; PEG-30 glyceryl laurate, Tagat® L2 (Goldschmidt), HLB 16; PEG-15 glyceryl laurate, Glycerox L series (Croda), HLB 15; PEG-40 glyceryl laurate, Glycerox L series (Croda), HLB 15; PEG-20 glyceryl stearate, Capmul® EMG (ABITEC), Aldo® MS-20 KFG (Lonza), HLB 13; PEG-20 glyceryl oleate, Tagat® 0 (Goldschmidt), HLB>10; PEG-30 glyceryl oleate, Tagat® 02 (Goldschmidt), HLB>10.

A large number of surfactants of different degrees of hydrophobicity or hydrophilicity can be prepared by reaction of alcohols or polyalcohols with a variety of natural and/or hydrogenated oils. Alcohol-oil transesterification products as embodiments of surfactant compositions of the present invention include, but are not limited to, PEG-3 castor oil, Nikkol CO-3 (Nikko), HLB 3; PEG-5, 9, and 16 castor oil, ACCONON CA series (ABITEC), HLB 6-7; PEG-20 castor oil, Emalex C-20 (Nihon Emulsion), Nikkol CO-20 TX (Nikko), HLB 11; PEG-23 castor oil, Emutgante EL 23, HLB>10; PEG-30 castor oil, Emalex C-30 (Nihon Emulsion), Alkamuls® EL 620 (Rhone-Poulenc), Incrocas 30 (Croda), HLB 11; PEG-35 castor oil, Cremophor EL and EL-F (BASF), Emulphor EL, Incrocas-35 (Croda), Emulgin RO 35 (Henkel); PEG-38 castor oil, Emulgante EL 65 (Condea); PEG-40 castor oil, Emalex C-40 (Nihon Emulsion), Alkamuls® EL 719 (Rhone-Poulenc), HLB 13; PEG-50 castor oil, Emalex C-50 (Nihon Emulsion), HLB 14; PEG-56 castor oil, Eumulgin® PRT 56 (Pulcra SA), HLB>10; PEG-60 castor oil, Nikkol CO-60TX (Nikko), HLB 14; PEG-I00 castor oil, Thomley, HLB>10; PEG-200 castor oil, Eumulgin® PRT 200 (Pulcra SA), HLB>10; PEG-5 hydrogenated castor oil, Nikkol HCO-5 (Nikko), HLB 6; PEG-7 hydrogenated castor oil, Simusolg 989 (Seppic), Cremophor WO7 (BASF), HLB 6; PEG-10 hydrogenated castor oil, Nikkol RCO-10 (Nikko), HLB 6.5; PEG-20 hydrogenated castor oil, Nikkol NCO-20 (Nikko), HLB 11; PEG-25 hydrogenated castor oil, Simulsol(D 1292 (Seppic), Cerex ELS 250 (Auschem SpA), HLB 11; PEG-30 hydrogenated castor oil, Nikkol HCO-30 (Nikko), HLB 11; PEG-40 hydrogenated castor oil, Cremophor RH 40 (BASF), (Croda), Emulgin HRE 40 (Henkel), HLB 13; PEG-45 hydrogenated castor oil, Cerex ELS 450 (Auschem SpA), HLB 14; PEG 50 hydrogenated castor oil, Emalex HC-50 (Nihon Emulsion), HLB 14; PEG-60 hydrogenated castor oil, Nikkol HCO-60 (Nikko), Cremophor RH 60 (BASF), HLB 15; PEG-80 hydrogenated castor oil, Nikkol HCO-80 (Nikko), HLB 15; PEG-100 hydrogenated castor oil, Nikkol HCO-100 (Nikko), HLB 17; PEG-6 corn oil, Labrafil® M 2125 CS (Gattefosse), HLB 4; PEG-6 almond oil, Labrafil® M 1966 CS (Gattefosse), HLB 4; PEG-6 apricot kernel oil, Labrafil® M 1944 CS (Gattefosse), HLB 4; PEG-6 olive oil, Labrafil® M 1980 CS (Gattefosse), HLB 4; PEG-6 peanut oil, Labrafil® M 1969 CS (Gattefosse), HLB 4; PEG-6 hydrogenated palm kernel oil, Labrafil® 2130 BS (Gattefosse), HLB 4; PEG-6 palm kernel oil, Labrafil® 2130 CS (Gattefosse), HLB 4; PEG-6 triolein, Labrafil® M 2735 CS (Gattefosse), HLB 4; PEG-8 corn oil, Labrafil® WL 2609 BS (Gattefosse), HLB 6-7; PEG-20 corn glycerides, Crovol M40 (Croda), HLB 10; PEG-20 almond glycerides, Crovol A40 (Croda), HLB 10; PEG-25 trioleate, TAGAT® TO (Goldschmidt), HLB 11; PEG-40 palm kernel oil, Crovol PK-70, HLB>10; PEG-60 corn glycerides, Crovol M70 (Croda), HLB 15; PEG-60 almond glycerides, Crovol A70 (Croda), HLB 15; PEG-4 caprylic/capric triglyceride, Labrafac® Hydro (Gattefosse), HLB 4-5; PEG-8 caprylic/capric glycerides, Labrasol (Gattefosse), Labrafac CM 10 (Gattefosse), HLB>10; PEG-6 capryllic/capric glycerides, SOFTIGEN® 767 (Huls), Glycerox capric glycerides 767 (Croda), HLB 19; Lauroyl macrogol-32 glyceride, GELUCIRE 44/14 (Gattefosse), HLB 14; Stearoyl macrogol glyceride, GELUCIRE 50/13 (Gattefosse), HLB 13; Mono, di, tri, tetra esters of vegetable oils and sorbitol, SorbitoGlyceride (Gattefosse), HLB<10; Pentaerythrityl tetraisostearate, Crodamol PTIS (Croda), HLB<10; Pentaerythrityl disteareate, Albunol DS (Taiwan Surf.), HLB<10; Pentaerythrityl tetraoleate, Liponate PO-4 (Lipo Chem.), HLB<10; Pentaerythrityl tetrastearate, Liponate PS-4 (Lipo Chem.), HLB<10; Pentaerythrityl tetracaprylate/tetracaprate, Liponate PE-810 (Lipo Chem.), Crodamol PTC (Croda), HLB<10; Pentaerythrityl tetraoctanoate, Nikkol Pentarate 408 (Nikko).

Polyglycerol esters of fatty acids include, but are not limited to, Polyglyceryl-2 stearate, Nikkol DGMS (Nikko), HLB 5-7; Polyglyceryl-2 oleate, Nikkol DGMO (Nikko), HLB 5-7; Polyglyceryl-2 isostearate, Nikkol DGMIS (Nikko), HLB 5-7; Polyglyceryl-3 oleate, Caprol® 3GO (ABITEC), Drewpol 3-1-0 (Stepan), HLB 6.5; Polyglyceryl-4 oleate, Nikkol Tetraglyn 1-0 (Niko), HLB 5-7; Polyglyceryl-4 stearate, Nikkol Tetraglyn 1-S (Nikko), HLB 5-6; Polyglyceryl-6 oleate, Drewpol 6-1-0 (Stepan), Nikkol Hexaglyn 1-0 (Nikko), HLB 9; Polyglyceryl-10 laurate, Nikkol Decaglyn 1-L (Nikko), HLB 15; Polyglyceryl-10 oleate, Nikkol Decaglyn 1-O (Nikko), HLB 14; Polyglyceryl-10 stearate, Nikkol Decaglyn 1-S (Nikko), HLB 12; Polyglyceryl-6 ricinoleate, Nikkol Hexaglyn PR-15 (Nikko), HLB>8; Polyglyceryl-10 linoleate, Nikkol Decaglyn 1-LN (Nikko), HLB 12; Polyglyceryl-6 pentaoleate, Nikkol Hexaglyn 5-O (Nikko), HLB<10; Polyglyceryl-3 dioleate, Cremophor GO32 (BASF), HLB<10; Polyglyceryl-3 distearate, Cremophor GS32 (BASF), HLB<10; Polyglyceryl-4 pentaoleate, Nikkol Tetraglyn 5-O (Nikko), HLB<10; Polyglyceryl-6 dioleate, Caprol® 6G20 (ABITEC); Hodag PGO-62 (Calgene), PLUROL OLEIQUE CC 497 (Gattefosse), HLB 8.5; Polyglyceryl-2 dioleate, Nikkol DGDO (Nikko), HLB 7; Polyglyceryl-10 trioleate, Nikkol Decaglyn 3-O (Nikko), HLB 7; Polyglyceryl-10 pentaoleate, Nikkol Decaglyn 5-O (Nikko), HLB 3.5; Polyglyceryl-10 septaoleate, Nikkol Decaglyn 7-O (Nikko), HLB 3; Polyglyceryl-10 tetraoleate, Caprol® 10G40 (ABITEC), Hodag PGO-62 (CALGENE), Drewpol 104-O (Stepan), HLB 6.2; Polyglyceryl-10 decaisostearate, Nikkol Decaglyn 1o-IS (Nikko), HLB<10; Polyglyceryl-101 decoaleate, Drewpol 10-10-0 (Stepan), Caprol® 10G100 (ABITEC), Nikkol Decaglyn 10-0, HLB 3.5; Polyglyceryl-10 mono, dioleate, Caprol® PGE 860 (ABITEC), HLB 11; Polyglyceryl polyricinoleate, Polymuls (Henkel), HLB 3-20.

Esters of propylene glycol and fatty acids include, but are not limited to, Propylene glycol monocaprylate, Capryol 90 (Gattefosse), Nikkol Sefsol 218 (Nikko), HLB<10; Propylene glycol monolaurate, Lauroglycol 90 (Gattefosse), Lauroglycol FCC (Gattefosse), HLB<10; Propylene glycol oleate, Lutrol OP2000 (BASF), HLB<10; Propylene glycol myristate, Mirpyl, HLB<10; Propylene glycol monostearate, ADM PGME-03 (ADM), LIPO PGMS (Lipo Chem.), Aldo® PGHMS (Lonza), HLB 3-4; Propylene glycol hydroxy stearate, HLB<10; Propylene glycol ricinoleate, PROPYMULS (Henkel), HLB<10; Propylene glycol isostearate, HLB<10; Propylene glycol monooleate, Myverol P-06 (Eastman), HLB<10; Propylene glycol dicaprylate/dicaprate, Captex® 200 (ABITEC), Miglyol® 840 (Huls), Neobee® M-20 (Stepan), HLB>6; Propylene glycol dioctanoate, Captex® 800 (ABITEC), HLB>6; Propylene glycol caprylatelcaprate, LABRAFAC PG (Gattefosse), HLB>6; Propylene glycol dilaurate, HLB>6; Propylene glycol distearate, Kessco®, PGDS (Stepan), HLB>6; Propylene glycol dicaprylate, Nikkol Sefsol 228 (Nikko), HLB>6; Propylene glycol dicaprate, Nikkol PDD (Nikko), HLB>6.

Mixtures of propylene glycol fatty acid esters and glycerol fatty acid esters include, but are not limited to, Oleic, ATMOS 300, ARLACEL 186 (ICI), HLB 3-4; Stearic, ATMOS 150, HLB 3-4.

Mono- and diglycerides include, but are not limited to, Monopalmitolein (C16:1, (Larodan), HLB<10; Monoelaidin (C18:1), (Larodan), HLB<10; Monocaproin (C6), (Larodan), HLB<10; Monocaprylin, (Larodan), HLB<10; Monocaprin, (Larodan), HLB<10; Monolaurin, (Larodan), HLB<10; Glyceryl monomyristate (C14), Nikkol MGM (Nikko), HLB 3-4; Glyceryl monooleate (C18:1), PECEOL (Gattefosse), Hodag GMO-D, Nikkol MGO (Nikko), HLB 3-4; Glyceryl monooleate, RYLO series (Danisco), DIMODAN series (Danisco), EMULDAN (Danisco), ALDO®, MO FG (Lonza), Kessco GMO (Stepan), MONOMULS® series (Henkel), TEGIN O, DREWMULSE GMO (Stepan), Atlas G-695 (ICI), GMOrphic 80 (Eastman), ADM DMG-40, 70, and 100 (ADM), Myverol (Eastman), HLB 3-4; Glycerol monooleate/linoleate, OLICINE (Gattefosse), HLB 3-4; Glycerol monolinoleate, Maisine (Gattefosse), MYVEROL 18-92, Myverol 18-06 (Eastman), HLB 3-4; Glyceryl ricinoleate, Softigen® 701 (Huls), HODAG GMR-D (Calgene), ALDO® MR (Lonza), HLB 6; Glyceryl monolaurate, ALDO® MLD (Lonza), Hodag GML (Calgene), HLB 6.8; Glycerol monopalmitate, Emalex GMS-P (Nihon), HLB 4; Glycerol monostearate, Capmul® GMS (ABITEC), Myvaplex (Eastman), IMWITOR® 191 (Huls), Cutina GMS, Aldo® MS (Lonza), Nikkol MGS series (Nikko), HLB 5-9; Glyceryl monodioleate, Capmul® GMO-K (ABITEC), HLB<10; Glyceryl palnitic/stearic, CUTINA MD-A, ESTAGEL-G18, HLB<10; Glyceryl acetate, Lanegin® EE (Grunau GmbH), HLB<10; Glyceryl laurate, Imwitor® 312 (Huls0, Monomuls® 90-45 (Grunau GmbH), Aldo® MLD (Lonza), HLB 4; Glyceryl citrate/lactate/oleate/linoleate, Imwitorg 375 (Huls), HLB<10; Glyceryl caprylate, Imwitor® 308 (Huls), Capmul® MCMC8 (ABITEC), HLB 5-6; Glyceryl caprylate/caprate, Capmul® MCM (ABITEC); HLB 5-6; Caprylic acid mono, diglycerides, Imwitor® 988 (Huls), HLB 5-6; Caprylic/capric glycerides, Imwitor® 742 (Huls), HLB<10; Mono- and diacetylated monoglycerides, Myvacet® 9-45, Myvacet® 9-40, Myvacet® 9-08 (Eastman), Lamegin® (Grunau), HLB 3.8-4; Glyceryl monostearate, Aldo® MS, Arlacel 129 (ICI), LIPO GMS (Lipo Chem.), Imwitor® 191 (Huls), Myvaplex (Eastman), HLB 4.4; Lactic acid esters of mono, diglycerides, LAMEGIN GLP (Henkel); Dicaproin (C6), (Larodan), LB<10; Dicaprin (C10), (Larodan), HLB<10; Dioctanoin (C8), (Larodan), HLB<10; Dimyristin (C14), (Larodan), HLB<10; Dipalmitin (C16), (Larodan), HLB<10; Distearin, (Larodan), HLB<10; Glyceryl dilaurate (C12), Capmul® GDL (ABITEC), HBL 3-4; Glyceryl dioleate, Capmul® GDO (ABITEC), HBL 3-4; Glycerol esters of fatty acids, GELUCIRE 39/01 (Gattefosse), HLB 1; GELUCIRE 43/01, (Gattefosse), GELUCIRE 37/06 (Gattefosse), HLB 6; Dipalmitolein (C16:1), (Larodan) HLB<10; 1,2 and 1,3-diolein (C18:1), (Larodan), HLB<10; Dielaidin (C18:1), (Larodan), HLB<10; Dilinolein (C18:2), (Larodan), HLB<10.

Sterol and sterol derivatives include, but are not limited to, cholesterol, sitosterol, lanosterol, HLB<10; PEG-24 cholesterol ether, Solulan C-24 (Amerchol), HLB>10; PEG-30 cholestanol, Nikkol DHC (Nikko), HLB>10; Phytosterol, GENEROL series (Henkel); HLB<10; PEG-25 phyto sterol, Nikkol BPSH-25 (Nikko), HLB>10; PEG-5 soya sterol, Nikkol BPS-5 (Nikko) HLB<10; PEG-10 soya sterol, Nikkol BPS-10 (Nikko), HLB<10; PEG-20 soya sterol, Nikkol BPS-20 (Nikko) HLB<10; PEG-30 soya sterol, Nikkol BPS-30 (Nikko), HLB>10.

Polyethylene glycol sorbitan fatty acid esters surfactant compositions of include, but are not limited to, PEG-10 sorbitan laurate, Liposorb L-10 (Lipo Chem.), HLB>10; PEG-20 sorbitan monolaurate, Tween-20 (Atlas/ICI), Crillet 1 (Croda), DACOL MLS 20 (Condea), HLB 17; PEG-4 sorbitan monolaurate, Tween-21 (Atlas/ICI), Crillet 11 (Croda), HLB 13; PEG-80 sorbitan monolaurate, Hodag PSML-80 (Calgene); T-Maz 28, HLB>10; PEG-6 sorbitan monolaurate, Nikkol GL-1 (Nikko), HLB 16; PEG-20 sorbitan monopalmitate, Tween-40 (Atlas/ICI), Crillet 2 (Croda), HLB 16; PEG-20 sorbitan monostearate, Tween-60 (Atlas/ICI), Crillet 3 (Croda), HLB 15; PEG-4 sorbitan monostearate, Tween-61 (Atlas/ICI), Crillet 31 (Croda), HLB 9.6; PEG-8 sorbitan monostearate, DACOL MSS (Condea), HLB>10; PEG-6 sorbitan monostearate, Nikkol TS106 (Nikko), HLB 11; PEG-20 sorbitan tristearate, Tween-65 (Atlas/ICI), Crillet 35 (Croda), HLB 11; PEG-6 sorbitan tetrastearate, Nikkol GS-6 (Nikko), HLB 3; PEG-60 sorbitan tetrastearate, Nikkol GS-460 (Nikko), HLB 13; PEG-5 sorbitan monooleate, Tween-81 (Atlas/ICI), Crillet 41 (Croda), HLB 10; PEG-6 sorbitan monooleate, Nikkol TO-106 (Nikko), HLB 10; PEG-20 sorbitan monooleate, Tween-80 (Atlas/ICI), Crillet 4 (Croda), HLB 15; PEG-40 sorbitan oleate, Emalex ET 8040 (Nihon Emulsion), HLB 18; PEG-20 sorbitan trioleate, Tween-85 (Atlas/ICI), Crillet 45 (Croda), HLB 11; PEG-6 sorbitan tetraoleate, Nikkol GO-4 (Nikko), HLB 8.5; PEG-30 sorbitan tetraoleate, Nikkol GO-430 (Nikko), HLB 12; PEG-40 sorbitan tetraoleate, Nikkol GO-440 (Nikko), HLB 13; PEG-20 sorbitan monoisostearate, Tween-120 (Atlas/ICI), Crillet 6 (Croda), HLB>10; PEG sorbitol hexaoleate, Atlas G-1086 (ICI), HLB 10; PEG-6 sorbitol hexastearate, Nikkol GS-6 (Nikko), HBL 3.

Polyethylene glycol alkyl ethers include, but are not limited to, PEG-2 oleyl ether, oleth-2, Brij 92/93 (Atlas/ICI), HLB 4.9; PEG-3 oleyl ether, oleth-3, Volpo 3 (Croda), HLB<10; PEG-5 oleyl ether, oleth-5, Volpo 5 (Croda), HLB<10; PEG-10 oleyl ether, oleth-10, Volpo 10 (Croda), Brij 96/97 (Atlas/ICI), HLB 12; PEG-20 oleyl ether, oleth-20, Volpo 20 (Croda), Brij 98/99 (Atlas/ICI), HLB 15; PEG-4 lauryl ether, laureth-4, Brij 30 (Atlas/ICI), HLB 9.7; PEG-9 lauryl ether, HLB>10; PEG-23 lauryl ether, laureth-23, Brij 35 (Atlas/ICI), HLB 17; PEG-2 cetyl ether, Brij 52 (ICI), HLB 5.3; PEG-10 cetyl ether, Brij 56 (ICI), HBL 13; PEG-20 cetyl ether, Brij 58 (ICI), HBL 16; PEG-2 stearyl ether, Brij 72 (ICI), HBL 4.9; PEG-10 stearyl ether, Brij 76 (ICI), HLB 12; PEG-20 stearyl ether, Brij 78 (ICI), HBL 15; PEG-100 stearyl ether, Brij 700 (ICI), HBL >10.

Sugar esters include, but are not limited to, Sucrose distearate, SUCRO ESTER 7 (Gattefosse), Crodesta F-10 (Croda), HLB 3; Sucrose distearate/monostearate, SUCRO ESTER 11 (Gattefosse), Crodesta F-110 (Croda), HLB 12; Sucrose dipalmitate, HLB 7.4; Sucrose monostearate, Crodesta F-160 (Croda), HLB 15; Sucrose monopalmitate, SUCRO ESTER 15 (Gattefosse), HLB>10; Sucrose monolaurate, Saccharose monolaurate 1695 (Mitsubishi-Kasei), HLB 15.

Polyethylene glycol alkyl phenols include, but are not limited to, PEG-10-100 nonyl phenol, Triton X series (Rohm & Haas), Igepal CA series (GAF, USA), Antarox CA series (GAF, UK), HLB>10; PEG-15-100 octyl phenol ether series, Triton N series (Rohm & Haas), Igepal CO series (GAF, USA), Antarox CO series (GAF, UK), HLB>10.

Polyoxyethylene-polyoxypropylene block copolymer surfactant compositions include, but are not limited to, Poloxamer 105, 1=11, b=16, HLB 8; Poloxamer 108, a=46, b=16, HLB >10; Poloxamer 122, a=5, b=21, HLB 3; Poloxamer 123, a=7, b=21, HLB 7; Poloxamer 124, a=11, b=21, HLB>7; Poloxamer 181, a=3, b=30; Poloxamer 182, a=8, b=30, HLB 2, Poloxamer 183, a=10, b=30; Poloxamer 184, a=13, b=30; Poloxamer 185, a=19, b=30, Poloxamer 188, a=75, b=30, HLB 29; Poloxamer 212, a=8, b=35; Poloxamer 215, a=24, b=35; Poloxamer 217, a=52, b=35; Poloxamer 231, a=16, b -39; Poloxamer 234, a=22, b=39; Poloxamer 235, a=27, b=39; Poloxamer 237, a=62, b=39, HLB 24; Poloxamer 238, a=97, b=39; Poloxamer 282, a=10, b=47; Poloxamer 284, a=21, b=47; Poloxamer 288, a=122, b=47, HLB>10; Poloxamer 331, a=7, b=54, HBL 0.5; Poloxamer 333, a=20, b=54; Poloxamer 334, a=31, b=54; Poloxamer 335, a=38, b=54; Poloxamer 338, a=128, b=54; Poloxamer 401, a=6, b=67; Poloxamer 402, a=13, b=67; Poloxamer403, a=21, b=67; Poloxamer 407, a=98, b=67.

Sorbitan fatty acid esters include, but are not limited to, Sorbitan monolaurate, Span-20 (Atlas/ICI), Crill 1 (Croda), Arlacel 20 (ICI), HLB 8.6; Sorbitan monopalmitate, Span-40 (Atlas/ICI), Criss 2 (Croda), Nikkol SP-10 (Nikko, HLB 6.7; Sorbitan monooleate, Span-80 (Atlas/ICI), Crill4 (Croda), Crill 50 (Croda), HLB 4.3; Sorbitan monostearate, Span-60 (Atlas/ICI), Criss 3 (Croda), Nikkol SS-10 (Nikko); HLB 4.7; Sorbitan trioleate, Span-85 (Atlas/ICI), Criss 45 (Croda), Nikkol SO-30 (Nikko), HLB 4.3; Sorbitan sesquioleate, Arlacel-C (ICI), Criss 43 (Croda), Nikkol S0-15 (Nikko), HLB 3.7; Sorbitan tristearate, Span-65 (Atlas/ICI), Crill 35 (Croda), Nikkol SS-30 (Nikko), HLB 2.1; Sorbitan monoisostearate, Crill 6 (Croda), Nikkol SI-10 (Nikko), HLB 4.7; Sorbitan sesquisterate Nikkol SS-15 (Nikko), HLB 4.2.

Lower alcohol fatty acid esters include, but are not limited to, Ethyl oleate, Crodamol EO (Croda), Nikkol EOO (Nikko), HLB<10; Isopropyl myristate, Crodamol IPM (Croda), HLB<10; Isopropyl palmitate Crodamol IPP (Croda), HLB<10; Ethyl linoleate, Nikkol VF-E (Nikko), HLB<10; Isopropyl linoleate, Nikkol VF-IP (Nikko), HLB<10.

Ionic surfactants, including cationic, anionic and zwitterionic surfactants, include fatty acid salts, for example, HLB>10: Sodium caproate, Sodium caprylate, Sodium caprate, Sodium laurate, Sodium myristate, Sodium myristolate, Sodium palmitate, Sodium palmitoleate, Sodium oleate; HLB 18: Sodium ricinoleate, Sodium linoleate, Sodium linolenate, Sodium stearate, Sodium lauryl sulfate (dodecyl); HBL 40: Sodium tetradecyl sulfate, Sodium lauryl sarcosinate, Sodium dioctyl sulfosuccinate [sodium docusate (Cytec)]; bile salts, for example, HLB>10: Sodium cholate, Sodium taurocholate, Sodium glycocholate, Sodium deoxycholate, Sodium taurodeoxycholate, Sodium glycodeoxycholate, Sodium ursodeoxycholate, Sodium chenodeoxycholate, Sodium taurochenodeoxycholate, Sodium glyco cheno deoxycholate, Sodium cholylsarcosinate, Sodium N-methyl taurocholate; phospholipids, for example, Egg/Soy lecithin [Epikuron™ (Lucas Meyer), Ovothin™ (Lucas Meyer)]; Lyso egg/soy lecithin, Hydroxylated lecithin, Lysophosphatidylcholine, Cariolipin, Sphingomyelin, Phosphatidylcholine, Phosphatidyl ethanolamine, Phosphatidic acid, Phosphatidyl glycerol, Phosphatidyl serine; phosphoesters, for example, Diethanolammonium polyoxyethylene-10 oleyl ether phosphate, Esterification products of fatty alcohols or fatty alcohol ethoxylates with phosphoric acid or anhydride; carboxylates, for example, Ether carboxylates (by oxidation of terminal OH group of fatty alcohol ethoxylates), Succinylated monoglycerides [LAMEGIN ZE (Henkel)], Sodium stearyl fumarate, Stearoyl propylene glycol hydrogen succinate, Mono/diacetylated tartaric acid esters of mono- and diglycerides, Citric acid esters of mono-, diglycerides, Glyceryl-lacto esters of fatty acids (CFR ref. 172.852), Acyl lactylates: lactylic esters of fatty acids, calcium/sodium stearoyl-2-lactylate, calcium/sodium stearoyl lactylate, Alginate salts, Propylene glycol alginate; sulfates and sulfonates, for example, Ethoxylated alkyl sulfates, Alkyl benzene sulfones, α-olefin sulfonates, Acyl isethionates, Acyl taurates, Alkyl glyceryl ether sulfonates, Octyl sulfosuccinate disodium; Disodium undecylenamideo-MEA-sulfosuccinate; CATIONIC Surfactants, for example, HLB>10: Hexadecyl triammonium bromide; Decyl trimethyl ammonium bromide; Cetyl trimethyl ammonium bromide; Dodecyl ammonium chloride; Alkyl benzyldimethylammonium salts; Diisobutyl phenoxyethoxydimethyl benzylammonium salts; Alkylpyridinium salts; Betaines (trialkylglycine); Lauryl betaine (N-lauryl,N,N-dimethylglycine); Ethoxylated amines: Polyoxyethylene-15 coconut amine.

The following examples are representative but not inclusive of the scope and conception of embodiments disclosed herein.

### EXAMPLES

### Example 1

Baseline and Comparative

IMITREX® Nasal Spray is comprised of an aqueous solution of sumatriptan having a concentration of 5 mg of sumatriptan in a 100-μL unit dose aqueous buffered solution containing monobasic potassium phosphate NF, anhydrous dibasic sodium phosphate USP, sulfuric acid NF, sodium hydroxide NF, and purified water USP. The pH of the solution is approximately 5.5. The osmolality of the solution is 372 mOsmol. In order to evaluate the uptake of Sumatriptan, a controlled study was done. IMITREX® Nasal Spray was first purchased from Glaxo Group Limited Corporation and was reformulated as follows for a comparative test.

| | Formula A | Formula B |
|---|---|---|
| Imitrex | 25 mL | 25 mL |
| a-Cyclodextrin | — | 5 gm |
| Benzalkonium chloride | 0.02 gm | 0.02 gm |
| Purified water q.s. | 100 mL | 100 mL |

After demonstrating that the dilutions were stable by HPLC for the active substance, and showed no precipitate or color change,

In preliminary tests, the material was permeated through a cartridge containing carefully standardized and viable mucosal respiratory endothelial cells (EpiAirway,). Each experiment lasted 2 hr. Surprisingly, the increased permeability was shown with the cyclodextrin containing formulation. Perhaps more surprisingly, the commercial formulation was relatively inert in the test and no evidence of meaningful active or passive uptake was shown. These data are summarized in the following table.

| | Uptake % | | Nasal |
|---|---|---|---|
| | over 2 hr | Tmax | irritation |
| Imitrex (diluted 1:8) | 1.8% | 1-1.5 hr | acceptable |
| Imitrex with | 34.0% | Not shown | acceptable |
| a-Cyclodextrin | | | |

Data were also collected in rats for nasal irritation. The results for both formulations were “acceptable”.

### Example 2

### Formulations S1, S2, S3, S4, S5, S18, S19, S20, S21.

The following formulations were then prepared from sumatriptan succinate obtained from Quimica Sintetica S.A.

| Component | S1 | S2 | S3 | S5 | S18 | S19 | S20 | S21 |
|---|---|---|---|---|---|---|---|---|
| Sumatriptan | 4% | 4% | 4% | 4% | 4% | 4% | 4% | 4% |
| Chitosan | 0.4% | | | | | | | |
| L-Arginine | | 12% | | | | 12% | 12% | |
| a-cyclodextrin | | | 5% | | 5% | 5% | | 5% |
| EDTA | | | | | | | 0.1% | 0.1% |

And the formulations were then tested in a small-scale tissue culture screening tool as described in Example 1.

| | S1 | S2 | S3 | S5 | S18 | S19 | S20 | S21 |
|---|---|---|---|---|---|---|---|---|
| Drug | 16% | 5% | 34% | 2% | 29% | 14% | 11% | 63% |
| Permeation | | | | | | | | |
| TEER | 50 | 251 | 2 | 2 | 29 | 44 | 32 | 0 |
| Viable Cells | 96% | 100% | 8% | 100% | 40% | 94% | 90% | 5% |

By way of explanation, higher drug permeation numbers indicate better passage of the drug substance through a model respiratory membrane. Many nasal drugs pass with 100% bioavailability, and score very well in this test. However, even limited uptake is sometimes acceptable when the drug can be successfully delivered at therapeutic concentrations by solubilization in a 0.1 mL dose volume. When solubility is limited, then uptake becomes critical.

TEER values refer to ohmmetry, where 600 mOhm is an approximate baseline, and treated cell membranes are hence more conductive, indicating that cellular tight junctions have been opened to current flow by the treatment.

Cell viability is recorded after 120 minutes. Dye reduction is a relatively accurate method of determining the extent of cell necrosis, more so perhaps than LDH release, which offers sometimes conflicting values after 2 hr, possibly because the released enzyme is subject to protease attack and denaturation.

### Example 3

### Microemulsion Oil Concentrate

The following formulations are prepared for intranasal administration:

| Ingredient | Wt or Vol | |
|---|---|---|
| Sumatriptan linolenate | 10 | mg |
| Transcutol | 30 | mg |
| Pyridoxine phosphate | 5 | mg |
| Riboflavin | 5 | mg |
| Phosphate buffer (10 mM) to pH 5 | q.s. 1 | mL |

| | |
|---|---|
| Sumatriptan linolenate | 10 mg |
| n,n-DMP | 35 mg |
| Lauryl-stearate | 10 mg |
| Transcutol | 35 mg |
| Propylene glycol monolaurate | 15 mg |
| Polyglyceryl-6 dioleate | 5 mg |
| Asparatyl-n-palmitate | 5 mg |
| Myristyl-carnitine | 5 mg |

### Example 4

Objective:

To improve absorption of Sumatriptan by adding enhancer/s to the marketed IMITREX® Nasal Spray.

IMITREX® Nasal Spray shows poor bioavailability in that Tₘₐₓ is achieved at 1-1.5 hours and Cₘₐₓ is very low. Various batches were prepared with different enhancers and tested via cell permeation study to verify the increase in drug permeation through the cell as compared to the current marketed product. Cell viability and transepithelial resistance were evaluated. Results showed that alpha cyclodextrin gave the best results in that cell permeation was 34% after 120 minutes vs. the control (IMITREX® Nasal Spray), which gave 1.8%.

Nasal irritation study was conducted in rats for 3 consecutive days using high (80 mg/g) and low (8 mg/g) dose formulations. Study showed acceptable results.

Materials/Method:

Complete Formulation of the Nastech Nasal Sumatriptan Preferred Formulation (hereinafter referred to as ‘Nastech Nasal’)

Sumatriptan Nasal Spray, 5.0 mg/0.1 g

(Modified from IMITREX® Nasal Spray 20 mg/0.1 mL)

| | | Concentration | Amount |
|---|---|---|---|
| # | Ingredients | % W/W | (g/10 g) |
| 1 | Imitrex ® (Sumatriptan) | 25.00 | 2.500 |
| | Nasal Spray (20 mg/0.1 mL) | | |
| 2 | Alpha cyclodextrin | 5.00 | 0.500 |
| 3 | Benzethonium Chloride, USP | 0.02 | 0.002 |
| 4 | Purified Water, USP q.s. to | 100.0 | 10.0 |

Method of Preparation:

A. Preparation of 6.7% Alpha-Cyclodextrin-0.027% Benzethonium Chloride Stock Solution:

| | | | Amount |
|---|---|---|---|
| # | Ingredients | % W/W | (g/5 g) |
| 1 | Alpha cyclodextrin | 6.70 | 0.335 |
| 2 | Benzethonium Chloride | 0.027 | 0.00135* |
| 3 | Purified Water, | 100.0 | 5.0 |
| | USP q.s. to | | |

*Since amount is too small for accurate weighing, it is recommended to prepare and use a 1% solution of Benzethonium Chloride.

1. Prepare 10.0 g of a 1% Benzethonium Chloride solution as follows:

a. Weigh a 20 mL glass beaker with a magnetic stirrer and record weight.

b. Weigh and add 0.100 g of Benzethonium Chloride into the beaker.

c. Add Purified Water to make up to 10.0 g. Mix until the solids are completely dissolved.

2. Prepare the alpha Cyclodextrin-Benzethonium Chloride stock solution as follows:

a. Weigh a 20 mL glass beaker with a magnetic stirrer and record weight.

b. Weigh and add 0.335 g of alpha cyclodextrin

c. Add 4.2 g of Purified Water and mix until dissolved.

d. Add 0.135 g of the 1% Benzethonium Chloride solution and mix.

e. Make up the final weight of solution with Purified Water to 5.0 g.

f. Fill the bulk in a 5 mL amber glass bottle.

B. Collection of IMITREX® 20 mg/0.1 g Solution:

1. Weigh a 4-mL Type 1 amber glass vial and record weight.

2. Take 10 units of IMITREX® 20 mg/0.1 mL Nasal Spray. Dispense and collect the contents into the small glass vial.

3. Take the gross weight and determine the net weight of IMITREX® 20 mg/0.1 mL Nasal Spray obtained.

4. Set aside the pooled sample to be mixed with the alpha cyclodextrin-Benzethonium Chloride stock solution.

C. Mixing of Preparation A (alpha cyclodextrin-Benzethonium Chloride stock solution) and Preparation B (IMITREX 20 mg/0.1 g solution)

(for a 4 g batch):

1. Weigh an 8 mL glass vial with a small magnetic stirrer and record weight.

2. Transfer accurately 1.00 g of the collected IMITREX® solution into the vial.

3. With stirring, add 3.00 g of alpha cyclodextrin-Benzethonium Chloride solution. Mix for 5 minutes.

Note: Proportion is 1 part Imitrex 20 mg/0.1 mL Nasal Spray to 3 parts diluent

4. Take the pH. Record color and clarity of the solution.

5. Fill the solution in a 5 mL amber bottle. Store at room temperature (15-30° C). Protect from light.

Stability Tests:

A. Stability tests on the complete formulation:

A batch was prepared wherein IMITREX® was diluted with the solution containing alpha cyclodextrin and benzethonium chloride. The batch was packaged in 4 mL Qorpak™clear vials with 1 mL fill per vial and kept at 40° C. and 25° C. for physical stability tests and assay for Sumatriptan.

Results of stability tests are shown in the table below.

| | | | | | | Assay for |
|---|---|---|---|---|---|---|
| Time point/ | | | Clar- | | Assay for | Benzethonium |
| Condition | | Color | ity | pH | Sumatriptan | Chloride |
| Initial | | yellow | clear | 5.65 | 102.2% | ND |
| 1 week | 40° C. | yellow | clear | 5.69 | 102.0% | ND |
| 2 | 25° C. | yellow | clear | 5.65 | 102.0% | ND |
| weeks | 40° C. | yellow | clear | 5.63 | 102.1% | ND |
| 20 | 25° C. | yellow | clear | 5.66 | 101.4% | 95.5% |
| weeks* | | | | | | |

ND: not determined
*excess sample after initial testing was set aside at 25° C. and tested after 20 weeks

The complete formulation is physically and chemically stable for at least 20 weeks at 25° C.

### Example 5

### Clinical Trial Report

A pilot bioavailability and tolerance study of intranasal sumatriptan formulation of the present invention described in Example 3, and commercially the intranasal sumatriptan formulation IMITREX® in healthy humans was conducted as described below.

### Introduction and Background

Sumatriptan is a selective agonist of vascular serotonin type 1 receptors, i.e., the 5-HT(1D) and 5-HT(1B) subtypes, and is structurally and pharmacologically related to serotonin. Sumatriptan may be effective for migraine attacks through selective constriction of certain large cranial blood vessels and inhibition of neurogenic inflammatory processes in the CNS. Indirect evidence suggests that serotonin is involved in the pathogenesis of migraine because of serotonin's physiologic effects and vasoconstriction. Serotonin levels within the vascular system have been shown to increase before and decrease rapidly after a migraine attack. In addition, urinary excretion of 5-hydroxyindoleacetic acid, a metabolite of serotonin, has been found in patients with migraine, suggesting a decrease in serotonin with migraine attacks.

There are three dosage forms of sumatriptan on the market to treat migraine attacks. Subcutaneous injectable Imitrex® (GlaxoSmithKline) is indicated for the acute treatment of migraine attacks with or without aura and the acute treatment of cluster headache episodes. Imitrex® tablets and nasal spray are indicated for the acute treatment of migraine attacks with or without aura.

Pharmacokinetic data from a single 6 mg subcutaneous dose to 18 healthy males is reported to have a mean Cₘₐₓ of 74±15 ng/ml and a median tₘₐₓ of 12 minutes with a range of 5 to 20 minutes. In a study of 20 females, the mean Cₘₐₓ from 5 and 20 mg intranasal doses was reported to be 5 and 16 ng/ml, respectively. The median tₘₐₓ was approximately 1 to 1.75 hours. The mean Cₘₐₓ is approximately 18 ng/ml, following oral dosing with 25 mg, with a tₘₐₓ of approximately 2 hours. The elimination half-life has been reported to be approximately 2 to 2.5 hours.

The onset of action of sumatriptan in patients with migraine or cluster headaches correlates with the peak plasma drug concentration. The therapeutic range in patients with migraine has ranged from 8 to 66 ng/ml. Onset of pain relief occurs approximately 10-34 minutes after a subcutaneous dose, 1 to 3 hours after an oral dose, and 30 minutes after a nasal dose.

Some migraine patients fear subcutaneous injections or tolerate them poorly and absorption from oral tablets may be erratic because of migraine-related vomiting or gastric stasis. The current Imitrex® nasal formulation has a tₘₐₓ of approximately 60 to 105 minutes. If the nasal formulation can be optimized to shorten the time to peak plasma levels then the onset of pain relief from migraine would be quicker.

### Study Objectives

The objective of the study was to determine how the absorption excipient (alpha-cyclodextrin) in the sumatriptan nasal formulation of the present invention described in Example 3 (hereinafter referred to as the Nastech formulation at a dose of 5 mg affects the bioavailability of sumatriptan compared to that obtained with the currently marketed reference products, Imitrex® Nasal Spray at a dose of 5 and 20 mg, and Imitrex® Oral Tablet at a dose of 25 mg.

### Study Design

This was a single center, crossover, open-label study to determine the nasal absorption tolerability and safety of the marketed Imitrex® products, given as a nasal spray and as an oral tablet, versus Nastech's nasal formulation in healthy male volunteers. A total of 12 qualified subjects were studied and received study medication. No placebo was used.

Enrolled subjects were administered the following products in the following manner: Visit 2-Imitrex® Nasal Spray Marketed Reference Product (5 mg/0.1 ml dose), Visit 3-Imitrex® Nasal Spray Marketed Reference Product (20 mg/0.1 ml dose), Visit 4-Imitrex® Oral Tablet Marketed Reference Product (25 mg dose), and Visit 5-Nastech's Nasal Formulation (5 mg/0.1 ml dose).

### Absorption and Pharmacokinetic Variables

All absorption data were plotted for individual subjects as well as for the averaged data. The Cₘₐₓ, tₘₐₓ, K01_{1/2} (absorption rate half life), and the AUC (bioavailability) values of the study drugs were evaluated.

### Statistical Methods

Descriptive statistics and pharmacokinetic parameters were calculated using WinNonLin (Version 4.0, Pharsight Corporation, Mountain View, Calif.).

### Study Subjects

A total of 11 subjects were enrolled in the study.

### Pharmacokinetic Results

This study evaluated the pharmacokinetics of Nastech's nasal formulation of sumatriptan versus the marketed nasal and oral Imitrex® products in healthy male volunteers. Pharmacokinetic calculations were performed using WinNonLin (Pharsight Corporation, Version 4.0, Mountain View, Calif.). The pharmacokinetic method used was a non-compartmental model.

12.1 Pharmacokinetic Variables

For each subject the following pharmacokinetic parameters were calculated, whenever possible, based on the plasma concentration of sumatriptan.

TABLE 12-1
Pharmacokinetic Parameters

| | |
|---|---|
| Cₘₐₓ | Maximum observed concentration |
| tₘₐₓ | Time to maximum concentration |
| AUC₀₋ₜ | Area under the concentration-time |
| | curve from time 0 (prior to dosing) to time t, |
| | calculated by the linear trapezoidal rule, where t is |
| | the time point of the last |
| | measurable concentration |
| AUC_{0-∞} | Area under the concentration-time curve |
| | extrapolated to infinity, calculated using the formula: |
| | AUC_{0-∞} = AUC₀₋ₜ + Cₜ/Kₑ |
| | Where Cₜ is the last measurable concentration |
| | and Kₑ is the apparent terminal |
| | phase rate constant |
| K01_{1/2} | Absorption rate half-life |
| Kₑ | Apparent terminal phase rate constant, where Kₑ is |
| | the magnitude of the slope of the linear regression of the |
| | log concentration versus time profile during the |
| | terminal phase |
| t_{1/2} | Apparent terminal phase half-life (whenever possible), |
| | where t_{1/2} = (In2)/Kₑ |

12.2 Plasma Sumatriptan Pharmacokinetic Results

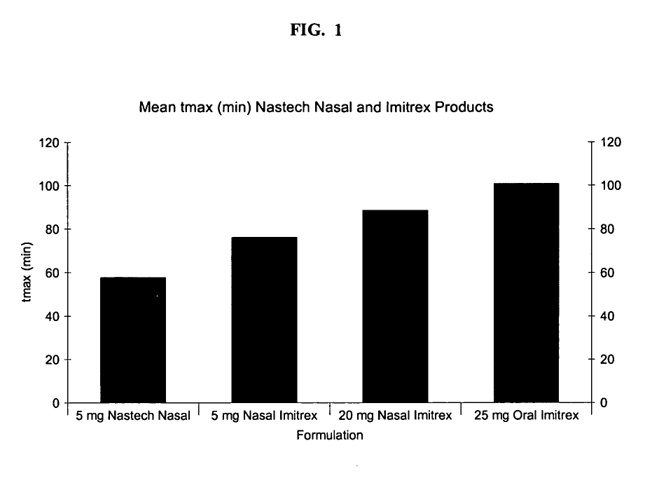

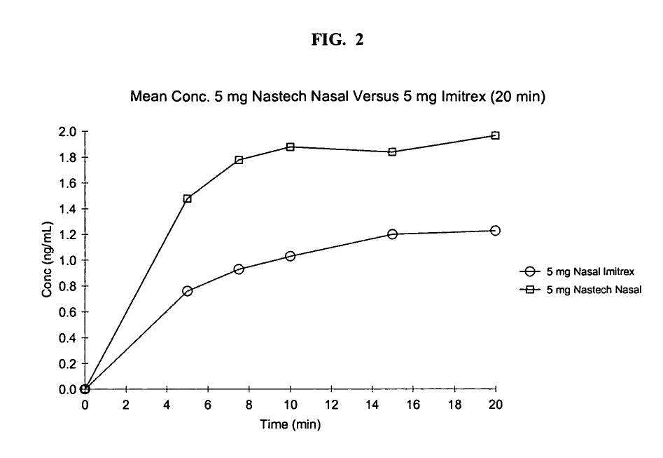

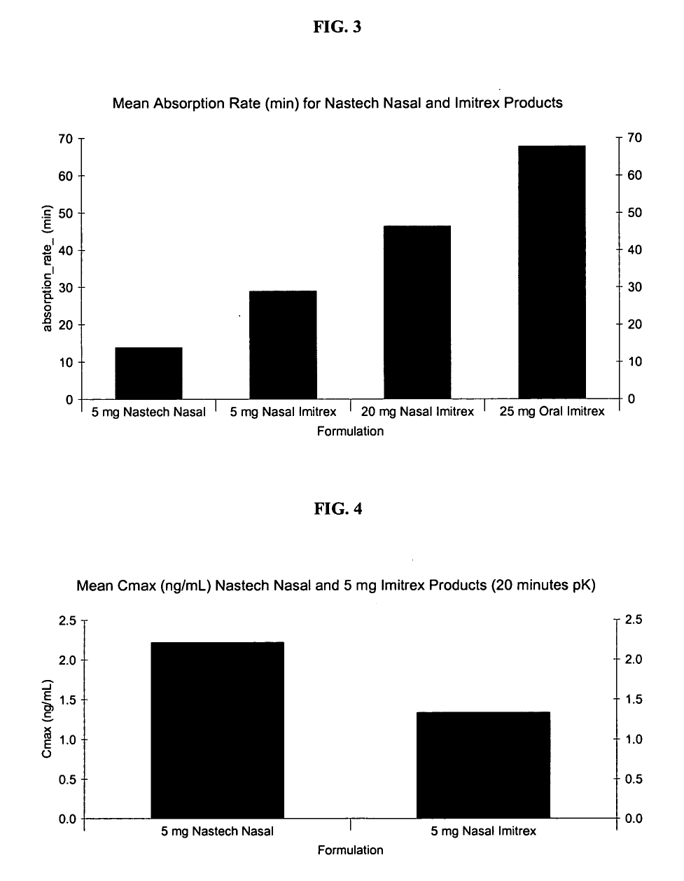

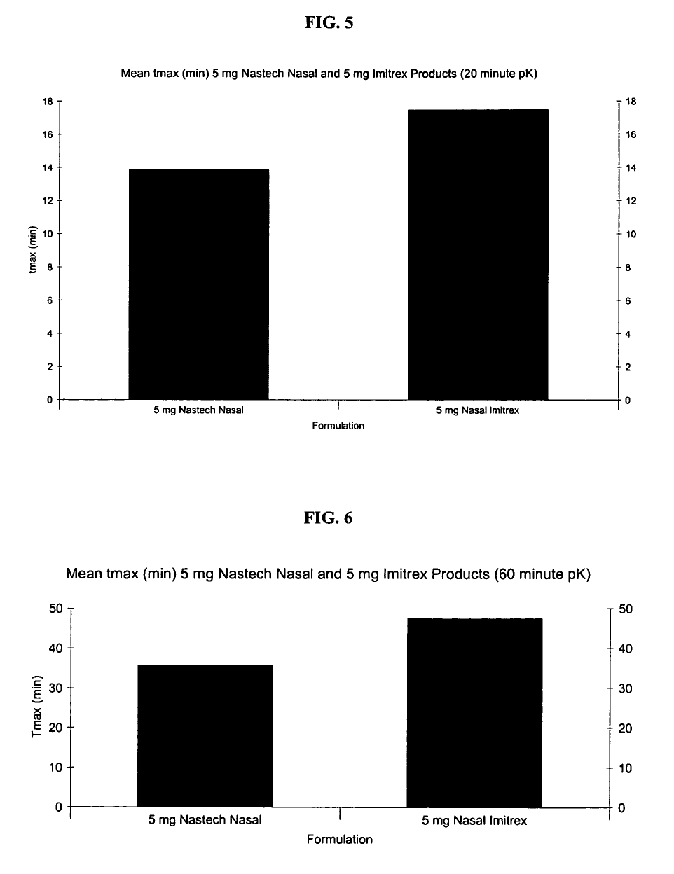

The mean tₘₐₓ (total area) for Nastech Nasal at 5 mg was 57.95 minutes and the mean tₘₐₓ for the marketed 5 mg nasal Imitrex® was 76.25 minutes. The mean tₘₐₓ (total area) values for the marketed 20 mg nasal Imitrex® and for the marketed 25 mg oral tablet were 88.64 and 100.91 minutes, respectively. The mean tₘₐₓ (total area) for the 4 formulations is presented in FIG. 1.

The mean partial areas under the curve for the first 20 minutes after dosing for Nastech's nasal formulation at 5 mg was 31.22 ng·min/ml and the marketed 5 mg nasal Imitrex® was 18.23 ng·min/ml. The mean AUCO_{0-∞} for the marketed 20 mg nasal Imitrexe and the marketed 25 mg oral tablet was 2380.35 and 2350.8 ng·min/ml, respectively

The mean terminal phase half-life (t_{1/2}) for the nasal and oral products was approximately 1.5 to 2 hours.

The mean absorption rate half-life (K01_{1/2}) for the Nastech nasal formulation at 5 mg was 13.9 minutes, indicating rapid absorption and the marketed 5 mg nasal Imitrex® was 28.97 minutes. The mean absorption rate half-life for the marketed 20 mg nasal Imitrex® and the marketed 25 mg oral tablet was 46.46 and 67.85 minutes, respectively.

The mean tₘₐₓ for the 20 and 60 minute PK analysis for the 5 mg nasal Imitrex® was 17.5 and 47.5 minutes, respectively. The mean tₘₐₓ for the 20 and 60 minute PK analysis for the 5 mg Nastech formulation was 13.86 and 35.68 minutes, respectively.

The mean Cₘₐₓ for the 20 minute PK analysis for the 5 mg nasal Imitrex®9 was 1.34. The mean Cₘₐₓ for the 20 PK analysis for the 5 mg Nastech formulation was 2.22.

The mean AUC₀₋ₜ for the 20 and 60 minute PK analysis for the 5 mg nasal Imitrex® was 18.24 and 95.46 ng·min/ml, respectively. The mean AUC₀₋ₜ for the 20 and 60 minute PK analysis for the 5 mg Nastech formulation was 30.32 and 107.55 ng·min/ml, respectively.

### Discussion and Overall Conclusions

Migraine is a neurological disorder that is characterized by recurrent attacks of headache, with pain most often on one side of the head, accompanied by various symptoms of nausea, vomiting, and sensitivity to light and sound. The disease is often hereditary and affects approximately 26 million Americans.

Serotonin (5-HT) receptor agonists have been shown to be effective in the treatment of acute migraines. Amerge® (naratriptan), Axert® (almotriptan), Frova® (frovatriptan), Maxalt® (rizatriptan), Zomig® (zolmitriptan) oral tablets and three dosage forms of Imitrex® (sumatriptan) are approved for the treatment of migraine.

There are three dosage forms of sumatriptan on the market to treat migraine attacks. Subcutaneous injectable Imitrex® is indicated for the acute treatment of migraine attacks with or without aura and the acute treatment of cluster headache episodes. Imitrex® tablets and nasal spray are indicated for the acute treatment of migraine attacks with or without aura.

Sumatriptan nasal spray has been proven to be useful for patients whose nausea and vomiting preclude them from using the oral migraine medication or for patients who prefer not to use an injectable migraine medication. In addition, the oral route's bioavailability is low, with only 14% of the administered drug reaching the systemic circulation because of first-pass metabolism.⁵

The commercially available nasal formulation of sumatriptan, Imitrexo Nasal Spray (marketed by GlaxoSmithKline) is an effective treatment for migraine. However, it has been reported that greater than 85% of the nasal dose is absorbed via the gastrointestinal route, therefore, also subjecting the drug to first-pass effect. A small amount of Imitrex® is absorbed by the nasal mucosa; approximately 0.5 mg of a 20 mg spray. However, the efficacy is similar to a 50 mg tablet, at about 50%, but less than a 6 mg subcutaneous dose that is almost 85% effective. The most common side effect of the Imitrex® nasal product is disturbance of taste that has been reported by almost 68 to 100% of patients taking the product. The bitter taste can worsen the nausea of the patient and precipitate vomiting. However, the Nastech nasal formulation of Example 3 was void of the unpleasant taste present in the IMITREX product.

The objective of the study was to determine how the absorption excipient (alpha-cyclodextrin) in Nastech's nasal formulation of sumatriptan at a dose of 5 mg affects the bioavailability of sumatriptan compared to that obtained with the currently marketed reference products, Imitrex® Nasal Spray at a dose of 5 and 20 mg and Imitrex® Oral Tablet at a dose of 25 mg. The comparisons are relevant when comparing the same nasal dosage strengths.

The full pK area mean tₘₐₓ for Nastech's nasal formulation was approximately 20 minutes shorter than the marketed product, when comparing the 5 mg doses. The onset of action of sumatriptan in patients with migraine headache correlates well with peak plasma drug concentration. Therefore, Nastech's nasal formulation should have a quicker onset of action, thus providing quicker headache relief.

In addition, the absorption rate half-life for the Nastech nasal formulation was 13.9 minutes, indicating rapid absorption compared to the marketed 5 mg nasal Imitrex® with an absorption rate half-life of 28.97 minutes. The mean absorption rate half-life for the marketed 20 mg nasal Imitrex® and the marketed 25 mg oral tablet was 46.46 and 67.85 minutes, respectively.

The partial areas under the curve for the first 20 minutes are greater for the Nastech 5 mg nasal sumatriptan formulation than for the 5 mg nasal marketed formulation, 31 min·ng/ml and 18 min·ng/ml, and the Cₘₐₓ is also greater; 2.22 versus 1.34 ng/ml, respectively. Partial areas under the curve for the first 60 minutes are also greater, however, the Cₘₐₓ is the same. The tₘₐₓ for Nastech's nasal formulation was approximately 4 minutes shorter within the first 20 minutes and 12 minutes shorter for the first 60 minutes after dosing. This would indicate that a greater amount of the product is being absorbed nasally than via the gastrointestinal tract for Nastech's nasal formulation as compared to the marketed Imitrex® formulation.

It has been reported that the 20 mg nasal dose of Imitrex® has a faster onset of action and similar symptom relief to that given by a 50 mg sumatriptan tablet, even if the plasma concentrations are half of that obtained by the oral dose. Speculation is that the nasal administration results in direct delivery to intracranial target structures. Recent results of Nastech's Phase I studies show that nasal delivery results in preferential uptake in the CSF versus the parenteral and enteral routes. This is the most likely explanation why the nasal route is more efficacious than the oral route, even at half the blood levels. Nastech's formulation would likely have greater CSF uptake than the marketed formulation based on the results of this study.

Bitter taste, burning throat, and difficulties in swallowing seem to be the most common side effects reported in subjects taking nasal Imitrex®. The literature reports anywhere from 68 to 100% of subjects report bitter taste. In our study, the incidence of bitter taste for Nastech's 5 mg formulation was 54.6% versus the 5 mg marketed product at 41.7%. However, the duration of bitter or unusual taste was 16 minutes for Nastech's formulation versus 41 minutes for the marketed Imitrex® at the same dose. This would have to be further investigated to determine if alpha-cyclodextrin in Nastech's formulation contributes to any masking of the bitter taste.

This study demonstrated that Nastech's Nasal sumatriptan formulation of Example 3 has a quicker absorption rate, greater nasal absorption, and less gastrointestinal absorption than the marketed nasal Imitrex® products.

What is claimed is:

1. An aqueous triptan formulation suitable for intranasal administration of a triptan comprised of a triptan, water, and an absorption enhancer wherein the triptan formulation has a tmax in serum of less than 15 minutes after intranasal administration of a sufficient amount of the formulation to deliver 5 mg of the triptan to the nose.

2. The aqueous triptan formulation of claim 1 wherein the triptan is selected from the group consisting of naratriptan, almotriptan, frovatriptan, rizatriptan, zolmitriptan, eletriptan and sumatriptan.

3. The aqueous triptan formulation of claim 1 wherein the absorption enhancer is a cyclodextrin.

4. The aqueous triptan formulation of claim 3 wherein the absorption enhancer is α-cyclodextrin.

5. The aqueous triptan formulation of claim 3 wherein the triptans are selected from the group consisting of naratriptan, almotriptan, frovatriptan, rizatriptan, zolmitriptan, eletriptan and sumatriptan.

6. The aqueous triptan formulation of claim 5 wherein the triptan present in the formulation is sumatriptan.

7. The aqueous triptan formulation of claim 6 further comprised of a preservative.

8. The aqueous triptan formulation of claim 1 wherein the absorption enhancer is chitosan.

9. The aqueous triptan formulation of claim 6 wherein the sumatriptan is present in the aqueous formulation at a concentration of about 4% weight/weight, and the alpha-cyclodextrin is present at a concentration of about 5% wt/wt.

10. The aqueous triptan formulation of claim 9 further comprised of a chelating agent.

11. The aqueous triptan formulation of claim 6 wherein the sumatriptan is present in the aqueous formulation at a concentration of about 25% wt/wt and the alpha-cyclodextrin is present at a concentration of about 5% wt/wt.

12. The aqueous triptan formulation of claim 1 wherein the absorption enhancer is chitosan.

13. An aqueous triptan formulation suitable for intranasal administration of a triptan comprised of a triptan, water, and an absorption enhancer wherein the triptan reaches a mean plasma concentration of at least 1.5 ng of triptan per mL of plasma within 20 minutes after intranasal administration of a sufficient amount of the formulation to deliver 5 mg of the triptan to the nose.

14. The aqueous triptan formulation of claim 12 wherein the triptan is selected from the group consisting of naratriptan, almotriptan, frovatriptan, rizatriptan, zolmitriptan, eletriptan and sumatriptan.

15. The aqueous triptan of claim 13 wherein the absorption enhancer is a cyclodextrin.

16. The aqueous triptan formulation of claim 15 wherein the absorption enhancer is α-cyclodextrin.

17. The aqueous triptan formulation of claim 12 wherein the triptan reaches a mean plasma concentration of at least 1.8 ng of triptan per milliliter of plasma within 20 minutes after intranasal administration of a sufficient amount of the formulation to deliver 5 mg of the triptan to the nose.

18. The aqueous triptan formulation of claim 14 wherein the triptan is selected from the group consisting of naratriptan, almotriptan, frovatriptan, rizatriptan, zolmitriptan, eletriptan and sumatriptan.

19. The aqueous triptan formulation of claim 12 wherein the triptan reaches a mean plasma concentration of at least 2.0 ng of triptan per milliliter of plasma within 20 minutes after intranasal administration of a sufficient amount of the formulation to deliver 5 mg of the triptan to the nose.

20. The aqueous triptan formulation of claim 16 wherein the triptan is selected from the group consisting of naratriptan, almotriptan, frovatriptan, rizatriptan, zolmitriptan, eletriptan and sumatriptan.

21. An aqueous triptan formulation suitable for intranasal administration of a triptan comprised of a triptan, water, and an absorption enhancer wherein the triptan formulation has a mean partial areas under the curve for the first 20 minutes after intranasal administration of a sufficient amount of the formulation to deliver 5 mg of the triptan to the nose of at least 25 ng per minute per mL of serum.

22. The aqueous triptan formulation of claim 18 wherein the triptan is selected from the group consisting of naratriptan, almotriptan, frovatriptan, rizatriptan, zolmitriptan, eletriptan and sumatriptan.

23. The aqueous triptan formulation of claim 18 wherein the triptan formulation has a mean partial area under the curve for the first 20 minutes after intranasal administration of a sufficient amount of the formulation to deliver 5 mg of the triptan to the nose of at least 30 ng per minute per mL of serum.

24. The aqueous triptan formulation of claim 20 wherein the triptan is selected from the group consisting of naratriptan, almotriptan, frovatriptan, rizatriptan, zolmitriptan, eletriptan and sumatriptan.

25. An aqueous triptan formulation suitable for intranasal administration of a triptan comprised of a triptan, water, and an absorption enhancer wherein the triptan formulation has a mean absorption rate of less than 20 minutes after intranasal administration of a sufficient amount of the formulation to deliver 5 mg of the triptan to the nose.

26. The aqueous triptan formulation of claim 22 wherein the triptan is selected from the group consisting of naratriptan, almotriptan, frovatriptan, rizatriptan, zolmitriptan, eletriptan, and sumatriptan.

27. The aqueous triptan formulation of claim 22 wherein the triptan formulation has a mean absorption rate of less than 15 minutes after intranasal administration of 5 mg of the triptan.

28. An aqueous triptan formulation suitable for intranasal administration of a triptan comprised of a triptan, water, and an absorption enhancer wherein the triptan formulation has a mean Cmax of at least 1.5 ng of triptan per mL of serum 20 minutes after intranasal administration of a sufficient amount of the formulation to deliver 5 mg of the triptan to the nose.

29. An aqueous triptan formulation suitable for intranasal administration of a triptan comprised of water, a cyclodextrin and one or more triptans selected from the group consisting of naratriptan, almotriptan, frovatriptan, rizatriptan, zolmitriptan eletriptan and sumatriptan.

30. The aqueous triptan formulation of claim 29 wherein the cyclodextrin is alpha-cyclodextrin.

31. The aqueous triptan formulation of claim 29 further comprised of a preservative.

32. The aqueous triptan formulation of claim 29 further comprised of a chelating agent.

33. The aqueous triptan formulation of claim 32 wherein the chelating agent is ethylene diamine tetraacetic acid (EDTA).

34. The aqueous triptan formulation of claim 30 wherein the triptan is sumatriptan and is present in the aqueous formulation at a concentration of about 4% weight/weight, and the alpha-cyclodextrin is present at a concentration of about 5% wt/wt.

35. The aqueous triptan formulation of claim 30 wherein the sumatriptan is present in the aqueous formulation at a concentration of about 25% wt/wt and the alpha-cyclodextrin is present at a concentration of about 5% wt/wt.

36. An aqueous sumatriptan formulation comprised of water, sumatriptan and alpha-cyclodextrin.

37. A method of treating a migraine headache comprising intranasally administering an aqueous triptan formulation wherein the formulation is comprised of a triptan, water, and an absorption enhancer wherein the triptan formulation has a tmax in serum of the triptan of less than 15 minutes after intranasal administration of a sufficient amount of the formulation to deliver 5 mg of the triptan to the nose.

38. The method of claim 37 wherein the triptan is selected from the group consisting of naratriptan, almotriptan, frovatriptan, rizatriptan, zolmitriptan, eletriptan and sumatriptan.

39. The method of claim 37 wherein the absorption enhancer is a cyclodextrin.

40. The method of claim 39 wherein the absorption enhancer is α-cyclodextrin.

41. The method of claim 39 wherein the triptans are selected from the group consisting of naratriptan, almotriptan, frovatriptan, rizatriptan, zolmitriptan, eletriptan and sumatriptan.

42. The method of claim 41 wherein the triptan present in the formulation is sumatriptan.

43. The method of claim 42 wherein the formulation is further comprised of a preservative.

44. The method of claim 37 wherein the absorption enhancer is chitosan.

45. The method of claim 42 wherein the sumatriptan is present in the aqueous formulation at a concentration of about 4% weight/weight, and the absorption enhancer is alpha-cyclodextrin present at a concentration of about 5% wt/wt.

46. The method of claim 45 wherein the aqueous formulation is further comprised of a chelating agent.

47. The method of claim 42 wherein the sumatriptan is present in the aqueous formulation at a concentration of about 25% wt/wt and the absorption enhancer is alpha-cyclodextrin present at a concentration of about 5% wt/wt.

48. The method of claim 37 wherein the absorption enhancer is chitosan.

49. A method for treating a migraine headache comprised of intranasally administering to an individual an aqueous triptan formulation comprised of a triptan, water, and an absorption enhancer wherein the triptan reaches a mean plasma concentration of at least 1.5 ng of triptan per mL of plasma within 20 minutes after intranasal administration of a sufficient amount of the formulation to deliver 5 mg of the triptan to the nose.

50. The method of claim of claim 49 wherein the triptan is selected from the group consisting of naratriptan, almotriptan, frovatriptan, rizatriptan, zolmitriptan, eletriptan and sumatriptan.

51. The method of claim 49 wherein the absorption enhancer is a cyclodextrin.

52. The method of claim 51 wherein the cyclodextran is α-cyclodextrin.

53. The method of claim 49 wherein the triptan reaches a mean plasma concentration of at least 1.8 ng of triptan per milliliter of plasma within 20 minutes after intranasal administration of a sufficient amount of the formulation to deliver 5 mg of the triptan to the nose.

54. The method of claim 53 wherein the triptan is selected from the group consisting of naratriptan, almotriptan, frovatriptan, rizatriptan, zolmitriptan, eletriptan and sumatriptan.

55. The method of claim 49 wherein the triptan reaches a mean plasma concentration of at least 2.0 ng of triptan per milliliter of plasma within 20 minutes after intranasal administration of a sufficient amount of the formulation to deliver 5 mg of the triptan to the nose.

56. The method of claim 55 wherein the triptan is selected from the group consisting of naratriptan, almotriptan, frovatriptan, rizatriptan, zolmitriptan, eletriptan and sumatriptan.

57. A method for treating a migraine headache in an individual comprising intranasally administering to the individual an aqueous triptan formulation comprised of a triptan, water, and an absorption enhancer wherein the triptan formulation has a mean partial areas under the curve for the first 20 minutes after intranasal administration of a sufficient amount of the formulation to deliver 5 mg of the triptan to the nose of at least 25 ng per minute per mL of serum.

58. The method of claim 57 wherein the triptan is selected from the group consisting of naratriptan, almotriptan, frovatriptan, rizatriptan, zolmitriptan, eletriptan and sumatriptan.

59. The method of claim 57 wherein the triptan formulation has a mean partial area under the curve for the first 20 minutes after intranasal administration of a sufficient amount of the formulation to deliver 5 mg of the triptan to the nose of at least 30 ng per minute per mL of serum.

60. The method of claim 59 wherein the triptan is selected from the group consisting of naratriptan, almotriptan, frovatriptan, rizatriptan, zolmitriptan, eletriptan and sumatriptan.

61. A method for treating a migraine headache in an individual comprising intranasally administering to the individual an aqueous triptan formlation comprised of a triptan, water, and an absorption enhancer wherein the triptan formulation has a mean absorption rate of less than 20 minutes after intranasal administration of a sufficient amount of the formulation to deliver 5 mg of the triptan to the nose.

62. The method of claim 61 wherein the triptan is selected from the group consisting of naratriptan, almotriptan, frovatriptan, rizatriptan, zolmitriptan, eletriptan, and sumatriptan.

63. The method of claim 61 wherein the triptan formulation has a mean absorption rate of less than 15 minutes after intranasal administration of 5 mg of the triptan.

64. A method for treating a migraine headache in an individual comprising intranasally administering to the individual an aqueous triptan formulation comprised of a triptan, water, and an absorption enhancer wherein the triptan formulation has a mean Cmax of at least 1.5 ng of triptan per mL of serum 20 minutes after intranasal administration of a sufficient amount of the formulation to deliver 5 mg of the triptan to the nose.

65. A method for treating a migraine headache in an individual comprising intranasally administering to the individual an aqueous triptan formulation comprised of water, a cyclodextrin and one or more triptans selected from the group consisting of naratriptan, almotriptan, frovatriptan, rizatriptan, zolmitriptan eletriptan and sumatriptan.

66. The method of claim 65 wherein the cyclodextrin is alpha-cyclodextrin.

67. The method of claim 65 wherein the formulation is further comprised of a preservative.

68. The method of of claim 65 wherein the formulation is further comprised of a chelating agent.

69. The method of claim 68 wherein the chelating agent is ethylene diamine tetraacetic acid (EDTA).

70. The method of claim 65 wherein the triptan present in the formulation is sumatriptan at a concentration of about 4% weight/weight, and the cyclodextrin is alpha-cyclodextrin at a concentration of about 5% wt/wt.

71. The method of claim 65 wherein the triptan contained in the formulation is sumatriptan and is present in the aqueous formulation at a concentration of about 25% wt/wt and the cyclodextrin is alpha-cyclodextrin and is present at a concentration of about 5% wt/wt.

72. A method for treating a migraine headache in an individual comprising intranasally administering to the individual an aqueous sumatriptan formulation comprised of water, sumatriptan and alpha-cyclodextrin.

73. The method of claim 72 wherein the alpha-cyclodextrin is present in the formulation at a concentration of about 5% w/w.
